(19) United States
(12) Patent Application Publication
(10) Pub. No.: US 2018/0049690 A1
(43) Pub. Date: Feb. 22, 2018

(54) SLEEP TRACKING SYSTEMS, METHODS, AND DEVICES

(71) Applicant: Matthew Walker, Berkeley, CA (US)

(72) Inventor: Matthew Walker, Berkeley, CA (US)

(21) Appl. No.: 15/681,277

(22) Filed: Aug. 18, 2017

Related U.S. Application Data
(60) Provisional application No. 62/377,458, filed on Aug. 19, 2016.
(60) Provisional application No. 62/377,461, filed on Aug. 19, 2016.
(60) Provisional application No. 62/378,045, filed on Aug. 22, 2016.

(51) Int. Cl. A61B 5/00; A61B 5/11

(52) U.S. Cl. CPC A61B 5/4812; A61B 5/4815; A61B 5/4064; A61B 5/6803; A61B 5/6817; A61B 5/1118

(57) ABSTRACT

Provided are mechanisms and processes for tracking sleep. Devices may include an accelerometer configured to measure macro and micro sensitivity movement of head, movement of throb of vessels close to the ear, monitor changes in heart rate and pulse rate and blood pressure. Devices may also include a sensor configured to measure changes in middle inner ear muscle, wherein the sensor is a pressure sensor configured to measure ear drum pressure differentiation as a measure of middle ear muscle activity. Devices may further include an electrical sensor configured to measure voltage of electrical activity from brain and identify and measure brain wave patterns.

### CROSS-REFERENCE TO RELATED APPLICATIONS

This application claims the benefit under 35 U.S.C. §119(e) of U.S. Provisional Patent Application No. 62/377,458, filed on Aug. 19, 2016, US Provisional Patent Application No. 62/378,045, filed on Aug. 22, 2016, and U.S. Provisional Patent Application No. 62/377,461, filed on Aug. 19, 2016, which are incorporated herein by reference in its entirety for all purposes.

### TECHNICAL FIELD

The present disclosure relates to device, systems, and processes directed to tracking sleep of users of sleep tracking devices.

### DESCRIPTION OF RELATED ART

Some wearable devices may be capable of identifying sleep periods associated with users. However, conventional wearable devices that implement such sleep tracking are not able to distinguish between phases of sleep accurately, and are limited in their ability to utilize sleep data.

### SUMMARY

Provided are various mechanisms and processes relating to an intelligent assistive mobility device. Systems are disclosed herein that may include a tracking device configured to generate sleep tracking data. The tracking device may include an accelerometer configured to measure macro and micro sensitivity movement of a head, movement and throb of vessels close to an ear, monitor changes in heart rate, pulse rate, and blood pressure of a user. The tracking device may also include a sensor configured to measure changes in a middle ear muscle, wherein the sensor is a pressure sensor configured to measure ear drum pressure differentiation as a measure of middle ear muscle activity. The tracking device may further include an electrical sensor configured to measure a voltage of electrical activity from a brain, and identify and measure brain wave patterns. Systems may also include a processing device configured to identify at least one of a plurality of stages of sleep based on the sleep tracking data generated by the tracking device.

In various embodiments, the accelerometer, the sensor, and the electrical sensor are included in a housing having a shape configured based on an ear of a user. In some embodiments, the accelerometer is configured to be positioned adjacent to an ear of a user. In some embodiments, the sensor is a mechanical sensor, and the sensor is configured to be positioned adjacent to an ear of a user. In various embodiments, the electrical sensor is configured identify the brain wave patterns based on electrophysiological measurements. According to various embodiments, the plurality of stages of sleep include a first stage of sleep, a second stage of sleep, a third stage of sleep, a fourth stage of sleep, and rapid eye movement (REM) sleep. In some embodiments, the at least one of the plurality of stages of sleep is identified based, at least in part, on a plurality of measurements made by the accelerometer, the sensor, and the electrical sensor. In various embodiments, the at least one of the plurality of stages of sleep is identified based, at least in part, on a plurality of measurements made by the accelerometer, the sensor, and the electrical sensor, and a transition associated with another of the plurality of stages of sleep. According to various embodiments, the system further comprises a communications interface configured to transmit measurements made by the accelerometer, the sensor, and the electrical sensor to the processing device.

Also disclosed herein are devices that include an accelerometer configured to measure macro and micro sensitivity movement of a head, movement and throb of vessels close to an ear, monitor changes in heart rate, pulse rate, and blood pressure of a user. The devices may further include a sensor configured to measure changes in a middle ear muscle, wherein the sensor is a pressure sensor configured to measure ear drum pressure differentiation as a measure of middle ear muscle activity. The devices may also include an electrical sensor configured to measure a voltage of electrical activity from a brain, and identify and measure brain wave patterns.

In some embodiments, the accelerometer, the sensor, and the electrical sensor are included in a housing having a shape configured based on an ear of a user. In various embodiments, the accelerometer is configured to be positioned adjacent to an ear of a user. According to various embodiments, the sensor is a mechanical sensor, and wherein the sensor is configured to be positioned adjacent to an ear of a user. In some embodiments, the electrical sensor is configured identify the brain wave patterns based on electrophysiological measurements. In various embodiments, the electrical sensor comprises an array of sensing elements. According to various embodiments, the devices further include a communications interface configured to transmit measurements made by the accelerometer, the sensor, and the electrical sensor.

Further disclosed herein are methods that include measuring, using an accelerometer, macro and micro sensitivity movement of a head, movement and throb of vessels close to an ear, changes in heart rate, pulse rate, and blood pressure of a user. The methods may also include measuring, using a sensor, changes in a middle ear muscle, wherein the sensor is a pressure sensor that measures ear drum pressure differentiation as a measure of middle ear muscle activity. The methods may further include measuring, using an electrical sensor, a voltage of electrical activity from a brain. The methods may also include identifying, using the electrical sensor, brain wave patterns. The methods may further include identifying at least one stage of sleep from a plurality of stages of sleep based on the measurements.

In some embodiments, the plurality of stages of sleep comprises a first stage of sleep, a second stage of sleep, a third stage of sleep, a fourth stage of sleep, and rapid eye movement (REM) sleep. In various embodiments, the at least one of the plurality of stages of sleep is identified based, at least in part, on a plurality of measurements made by the accelerometer, the sensor, and the electrical sensor. According to various embodiments, the at least one of the plurality of stages of sleep is identified based, at least in part, on a plurality of measurements made by the accelerometer, the sensor, and the electrical sensor, and a transition associated with another of the plurality of stages of sleep.

This and other embodiments are described further below with reference to the figures.

### BRIEF DESCRIPTION OF THE DRAWINGS

FIG. 1 illustrates an example of a sleep tracking device, configured in accordance with some embodiments.

FIG. 2 illustrates an example of a sleep tracking system, configured in accordance with some embodiments.

FIG. 3 illustrates an example of a sleep tracking method, configured in accordance with some embodiments.

FIG. 4 illustrates an example of another sleep tracking method, configured in accordance with some embodiments.

FIG. 5 illustrates an example of yet another sleep tracking method, configured in accordance with some embodiments.

FIG. 6 illustrates an example of a processing device that can be used with various embodiments of the present invention.

### DETAILED DESCRIPTION OF EXAMPLE EMBODIMENTS

Reference will now be made in detail to some specific examples of the invention including the best modes contemplated by the inventors for carrying out the invention. Examples of these specific embodiments are illustrated in the accompanying drawings. While the present disclosure is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims. In addition, although many of the components and processes are described below in the singular for convenience, it will be appreciated by one of skill in the art that multiple components and repeated processes can also be used to practice the techniques of the present disclosure.

In the following description, numerous specific details are set forth in order to provide a thorough understanding of the present invention. Particular embodiments of the present invention may be implemented without some or all of these specific details. In other instances, well known process operations have not been described in detail in order not to unnecessarily obscure the present invention.

Although many people can benefit from wearable pedometers or fitness trackers, people with compromised physical abilities, such as aging adults or disabled persons, may need to use assistive devices such as walkers or wheelchairs to move around. These types of movements may not necessarily be trackable with currently available devices. Specifically, bracelets or bands that measure the cadence of a person's arm as they walk may not register movement or steps of someone who is holding onto the handles of a walker. However, these people may still be interested in improving and monitoring their health and fitness. Accordingly, various embodiments disclosed herein relate to improved mechanisms and processes for monitoring the health and fitness of people using assistive devices.

In some embodiments, billions of hours of sleep are being recorded from millions of users every night with various sleep tracking devices. However, sleep data generated by such devices (and individuals “sleep history”) is often not utilized or harnessed, and is not used to connect with industry or government, to improve the health, wellness, productivity of society and business. In various embodiments, sleep data from a user is integrated into a centralized database system which is configured as a personal hub. That database system is then utilized by industries (e.g., health care, such as HMOs or hospitals) or governments (e.g., military) of the individual's choosing. Those organizations then use the sleep data to back-inform the individual for utility purposes.

For example, if an individual is not getting more than 6-hours of sleep, on average, during the 5 days leading up to their flu shot (immunization for the influenza virus), the individual will produce less than half of the normal antibody response, rendering the vaccine markedly ineffective. In this example, the centralized database can provide the ongoing sleep average amounts of individuals to their HMO. Each time the individual his achieving a string of good nights of sleep during flu season, the HMO can send them a text or email telling then that now is a great time to come and get a flu shot because of the amount of sleep they have obtained. Additional examples of embodiments that may create a “utilitarian loop” between an individual user and an industry or government of choosing, and harness the sleep data information to bring about utility good and cost savings are discussed in greater detail below.

Furthermore, many sleep tracking devices are limited in their ability to accurately discern the different stages of sleep human sleep: non-rapid eye movement (NREM) sleep stages 1-4 (and 3&4 known as slow wave, or deep sleep), and rapid eye movement sleep (REM; named because of the eye movements that physically occur during this stage of sleep). The inaccuracy of current commercial sleep trackers is especially pronounced for REM sleep.

In various embodiments, an ear-related device (in-ear and/or surround ear) is used that senses middle ear muscle activity (MEMA) that coincides with the rapid eye movement of REM sleep, using electrical and pressure sensors. In addition, these sensors are configured to track an individual's pulse (vesicle “throb” related to a heartbeat) using electrical and blood vesicle movement activity. These signals (MEMAs, pulse) are further integrated with accelerometer device signals that further track the lowered or absence of muscle tone of the body (the rest of the skeletal muscles), also known as atonia, that occurs during REM sleep. Using just the MEMAs, the MEMAs in combination with the pulse, or MEMAs and pulse in combination with accelerometer signals, a plurality of parameters from the recorded measures will result in a data stream that is indicative of sleep stages—both REM and inference, NREM and wakefulness).

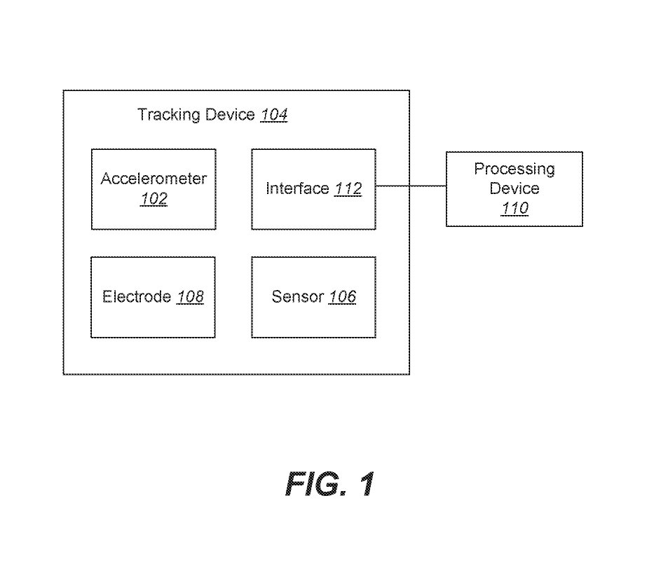

FIG. 1 illustrates an example of a sleep tracking device, configured in accordance with some embodiments. In various embodiments, a sleep tracking device, such as tracking device 104, may be configured to be positioned in a user's ear or surrounding the user's ear. For example, tracking device 104 may include a housing that may be configured to fit a user's ear. In some embodiments, the housing may be an in-ear bud, and tracking device 104 may be configured to fit securely within a user's ear. In various embodiments, the housing may be configured to fit around the user's ear, and tracking device 104 may be configured to encircle the user's ear.

In various embodiments, tracking device 104 may include accelerometer 102 which is configured to identify and measure various changes in a user's position that may be due to movement that occurs during sleep. For example, accelerometer may be configured to identify and measure lowered or absence of muscle tone of various muscles, such as skeletal muscles of the body. Moreover, accelerometer 102 may be configured to obtain various measurements based on movement and activity associated with blood vesicles in and surrounding the ear, and may be configured to acquire heart rate data. Accordingly, accelerometer 102 may be a multi-axis accelerometer that acquires one or more periodic measurements. In various embodiments, accelerometer 102 may be configured to be positioned in a user's ear or surrounding the user's ear. In some embodiments, such configurations are determined based on a design and configuration of tracking device 104 and a housing associated with tracking device 104. More specifically, tracking device 104 and an associated housing may be configured to position accelerometer 102 adjacent to a portion of a user's ear that facilitates the acquisition of several of the above-mentioned measurements, such as heart rate and muscle tone, simultaneously.

In some embodiments, tracking device 104 may also include sensor 106 which may be a mechanical or pressure sensor. Accordingly, sensor 106 may be configured to be positioned in a user's ear or surrounding the user's ear. As similarly discussed above, such configurations may be determined based on a design and configuration of tracking device 104 and a housing associated with tracking device 104. In various embodiments, sensor 106 may be configured to identify and measure middle ear muscle activity (MEMA). As similarly discussed above, MEMA may correlate with rapid eye movement of REM sleep. Accordingly, as will be discussed in greater detail below, measurements made by sensor 106 relating to MEMA may be used to identify and measure REM stages of sleep. In some embodiments, sensor 106 includes a pressure transducer that converts a raw force signal into an electrical signal which is acquired as a measurement. In various embodiments, the position of sensor 106 is configured based on the user's ear, and may also be positioned based on a location of accelerometer 102. For example, accelerometer 102 may be positioned in a first location of the user's ear, and sensor 106 may be positioned in a second location of the user's ear. In this example, the positioning of sensor 106 at the second location provides additional information about the user's sleep state from a different region of the user's ear.

Tracking device 104 may also include electrode 108 which may be configured to measure and acquire electrophysiological measurements. Such measurements may be used to identify brain activity of a user. Accordingly, electrode 108 may be configured to obtain measurements of the voltage of electrical activity from the user's brain, and may be configured to monitor and identify particular brain waves, and patterns of brain activity. More specifically, the electrophysiological measurements may identify and measure brain waves produced by neural activity such as alpha waves, theta waves, and delta waves. Such measurements may identify amplitudes and frequencies associated with such brain waves. While tracking device 104 is described above as including accelerometer 102, electrode 108, and sensor 106, embodiments disclosed herein contemplate that such components may include single elements, such as sensors, or multiple elements, such as several sensors. For example, electrode 108 may include an array of electrodes.

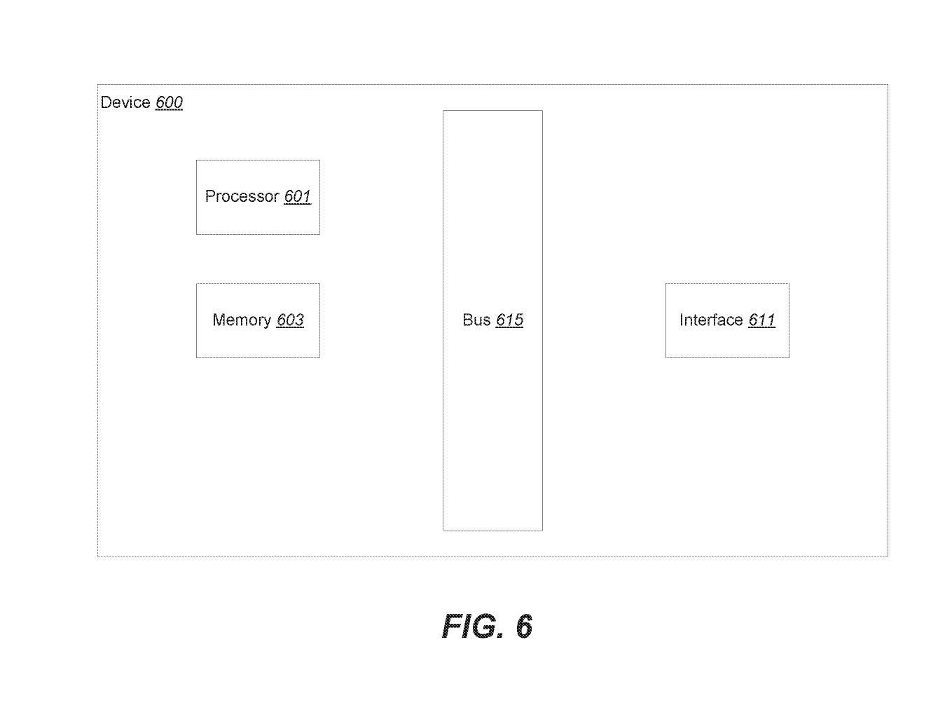

Tracking device 104 may be coupled with processing device 110 via a communications interface, such as interface 112. Interface 112 may be a wired or wireless connection, as may be enabled by Bluetooth or WiFi radio. In various embodiments, processing device 100 may include one or more components as discussed below with reference to FIG. 6, such as a processor, memory, and communications interface. In some embodiments, processing device 110 may be implemented in logic of one or more hardware components that enables mounting on a local and separate housing than that of tracking device 104. In one example, processing device 110 may be implemented as a local computing device, such as a local personal computer. Moreover, while processing device 110 is displayed as separate from tracking device 104, in some embodiments, processing device 110 is implemented within the housing of tracking device and an onboard processing device. Accordingly, various implementations and configurations of processing device 110 are contemplated and disclosed herein.

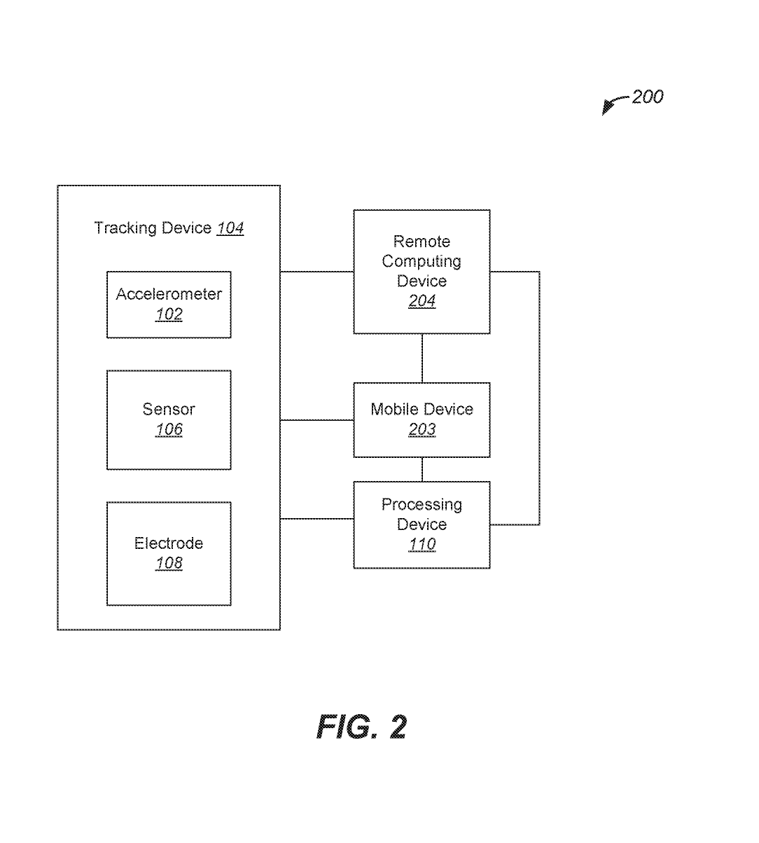

FIG. 2 illustrates an example of a sleep tracking system, configured in accordance with some embodiments. Accordingly, a tracking device, such as tracking device 104 discussed above, may be communicatively coupled with various different computer systems and databases via one or more connections and/or networks. As will be discussed in greater detail below, such connectivity enables the acquisition and utilization of sleep tracking data generated by tracking device 104.

In various embodiments, sleep tracking system 200 may include a sleep tracking device, such as tracking device 104. Accordingly, sleep tracking system 200 may include tracking device 104 and its associated components such as accelerometer 102, sensor 106, electrode 108, as well as one or more interfaces such as interface 112. As discussed above, tracking device 104 may be configured to obtain measurements relating to a user's heart rate, MEMAs, muscle tone, and muscular movement. Such data may be transmitted via interface 112 to various computing devices and systems as well as databases discussed in greater detail below.

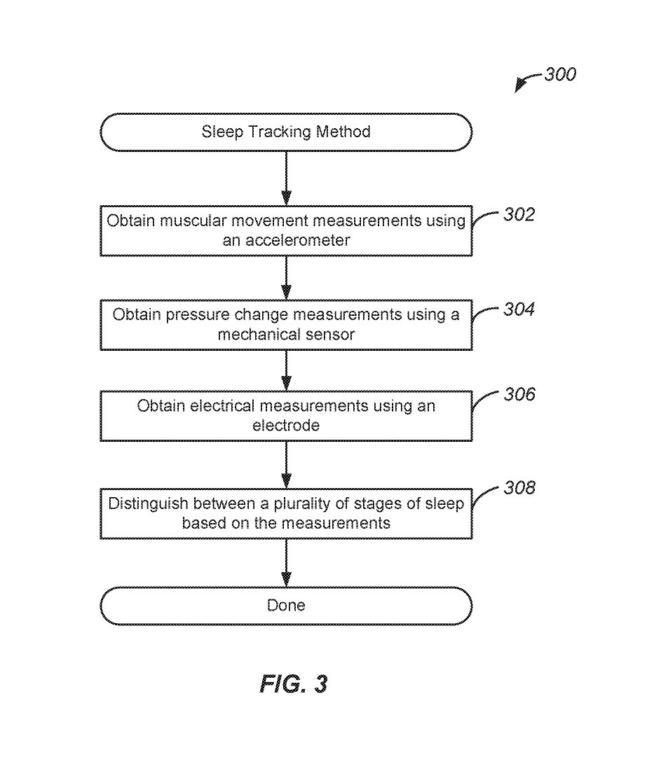

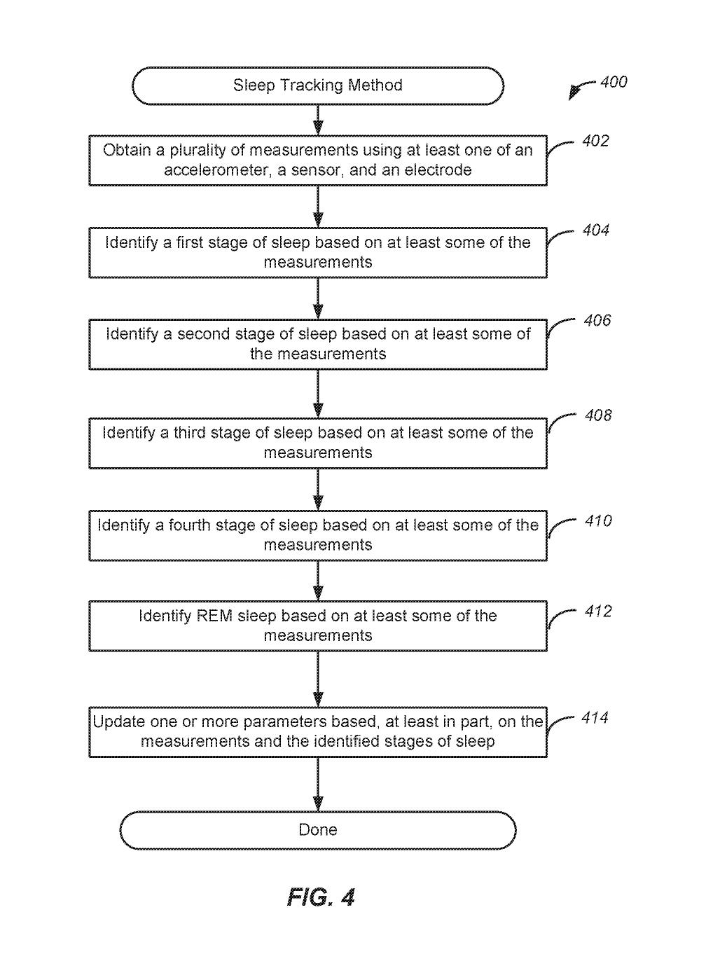

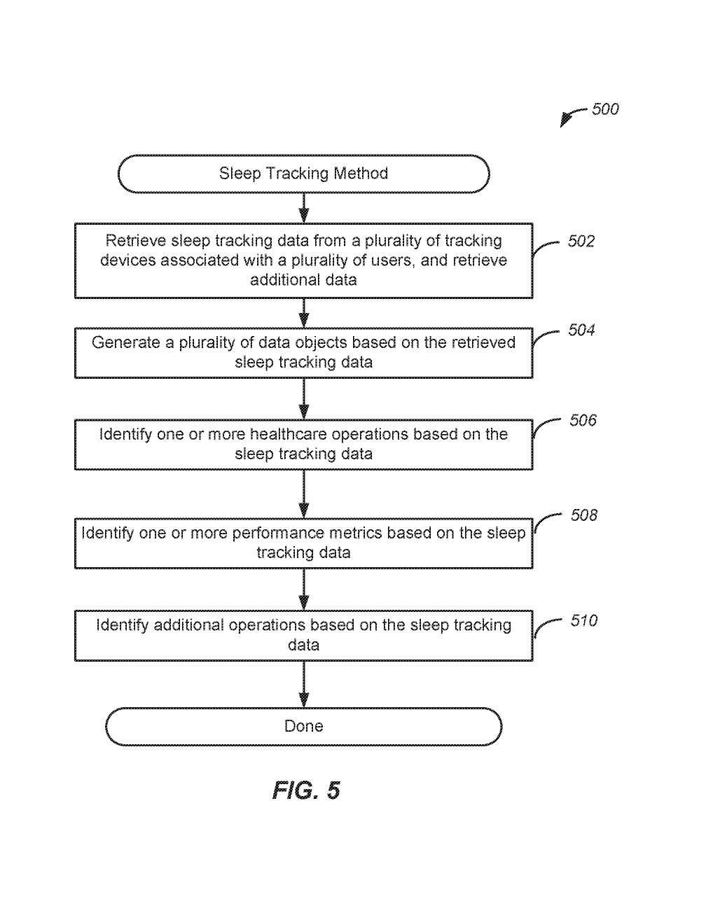

In some embodiments, sleep tracking system 200 includes one or more local computing devices, such as processing device 110. In various embodiments, processing device 110 may be implemented and configured as discussed above with reference to FIG. 1, and as discussed below with reference to FIG. 6. Accordingly, processing device 110 may be located adjacent to the user, such as in the user's home, and may be coupled to tracking device 104 via a wired connection or over a local network. In various embodiments, processing device 110 may be configured to perform various processing operations, such as those discussed below with reference to FIG. 3, FIG. 4, and FIG. 5. For example, processing device 110 may be the user's personal computer that is configured to receive sleep tracking data that includes measurement data from tracking device 104, and is further configured to perform one or more operations based on such data.

Sleep tracking system 200 may also include mobile device 203. In various embodiments, mobile device 203 may be a mobile phone or wearable device. Mobile device 203 may be coupled with tracking device 104 via a wireless communications network or other wireless network. In various embodiments, mobile device 203 may be configured to perform various processing operations, such as those discussed below with reference to FIG. 3, FIG. 4, and FIG. 5. Accordingly, mobile device 203 may be configured to receive data from receive sleep tracking data that includes measurement data from tracking device 104, and may be further configured to perform one or more operations based on such data.

Sleep tracking system 200 may further include remote computing device 204. In some embodiments, remote computing device 204 may be a computer system that is operated and maintained by an entity, such as an organization or company. For example, remote computing device 204 may be operated and maintained by a health maintenance organization (HMO), government, or other online service provider. Moreover, remote computing device 204 may be coupled with numerous tracking devices and may receive sleep tracking data from numerous different users. Thus, remote computing device 204 may be configured to receive data from potentially thousands to millions of users. In some embodiments, remote computing device 204 may be include one or more components discussed below with reference to FIG. 6 such as a processor, memory, and communications interface, and may be a computer system that is configured to receive large amounts of data from various different users, and is further configured to include or be coupled with a database system that stores such data. As similarly discussed above, remote computing device 204 may be coupled with tracking device 104 via a communications network or the internet.

FIG. 3 illustrates an example of a sleep tracking method, configured in accordance with some embodiments. As discussed above, a sleep tracking device, such as tracking device 104, may be configured to obtain various sleep tracking data, and such sleep tracking data may be analyzed to implement one or more operations. Accordingly, method 300 may commence with operation 302 during which an accelerometer may obtain muscular movement measurements. As discussed above, the accelerometer may obtain various measurements included in the sleep tracking data that identify and measure lowered or absence of muscle tone of various muscles, such as skeletal muscles of the body, as well as movement and activity associated with blood vesicles in and surrounding the ear, and may be configured to acquire heart rate data. In this way, the accelerometer may measure the movement of a user's head, the movement of throb of vessels close to the ear, and may monitor changes in heart rate and pulse rate and blood pressure.

Method 300 may proceed to operation 304 during which a sensor may obtain pressure change measurements. As discussed above, the sensor may identify and measure middle ear muscle activity (MEMA) which may correlate with rapid eye movement of REM sleep. For example, the sensor may measure changes in a middle or inner ear muscle and pressures generated by the middle inner ear muscle on an inserted earbud, or on a sensor around the ear depending on the configuration of the tracking device. The sensor may also measure ear drum pressure differentiation as a measure of middle ear muscle activity. As will be discussed in greater detail below, such measurements may be used to identify and measure REM stages of sleep.

Method 300 may proceed to operation 306 during which an electrode may obtain electrical measurements. As discussed above, the electrode may be used to obtain measurements of the voltage of electrical activity from the user's brain, and may be configured to monitor and identify particular brain waves, and patterns of brain activity. Such measurements may be made in the user's ear or surrounding the user's ear, depending on the configuration of the tracking device.

Method 300 may proceed to operation 308 during which a processing device may distinguish between a plurality of stages of sleep based on the obtained measurements. As will be discussed in greater detail below, the measurements, as well as combinations of the measurements, may be used to identify stages of sleep, and to distinguish between particular stages of sleep. Accordingly, as a user enters and continues to sleep, sleep tracking data may be gathered that identifies when a user entered each respective stage of sleep and for how long.

FIG. 4 illustrates an example of another sleep tracking method, configured in accordance with some embodiments. As discussed above, various sleep tracking data may be collected, and measurements included in the sleep tracking data, as well as combinations of the measurements, may be used to identify stages of sleep, and to distinguish between particular stages of sleep. Accordingly, method 400 may commence with operation 402 during which a plurality of measurements may be obtained using a tracking device, such as tracking device 104 discussed above. Accordingly, various measurements may be obtained using at least one of an accelerometer, a sensor, and an electrode while a user sleeps, and during the duration of the user's period of sleep. Such measurements and sleep tracking data may be streamed and transmitted to a processing device, or other component, such as a mobile device or a computing device.

Method 400 may proceed to operation 404 during which a first stage of sleep may be identified. In various embodiments, the first stage of sleep may be identified based on patterns in measurements indicative of light sleep. In some embodiments, the first stage may be identified based on muscular measurements, such as those made by an accelerometer and a sensor described above. For example, if the accelerometer identifies a slowing of muscular activity and/or the presence of muscular contractions associated with hypnic myoclonia, then the first stage may be identified. In one example, the first stage may be identified based on a particular increase, or spike, in the measurement associated with a hypnic jerk that causes a detected amount of an increase in measured signal over a designated period of time. In another example, the first stage is identified based on a combination of the measurements from the accelerometer and the measurements from the sensor. More specifically, the first stage is identified based on a combination of the detection of the previously described increase or spike as well as a corresponding increase in the measurement from the sensor. Moreover, such parameters associated with the measurements used to identify the first stage may be configured based on characteristics of the user. For example, thresholds associated with amplitudes and time intervals of measurements used to identify the first stage may be configured based on previously generated sleep tracking data generated by the user in previous sleep cycles during a learning phase of use of a tracking device. Alternatively, default values may be used.

Method 400 may proceed to operation 406 during which during which a second stage of sleep may be identified. In various embodiments, the second stage of sleep may be identified based on additional patterns in the measurements, such as muscular movements, such as eye movements, slowing or stopping, and brain waves slowing. Accordingly, the second stage may be identified based on a combination of muscular measurements and electrical measurements. For example, if movements detected by the accelerometer slow or stop and the electrical activity detected by the electrode slows, then the second stage may be identified. More specifically, the second stage is identified if movements detected by the accelerometer meet a designated frequency parameter, and activity measured by the electrode meets a designated frequency parameter, which may be a frequency threshold. Furthermore, identification of the second stage may also be dependent on the occurrence or detection of the first stage, and may occur after and responsive to the identification of the first stage. As similarly discussed above, the parameters may be configured based on previously generated sleep tracking data generated by the user in previous sleep cycles during a learning phase of use of a tracking device. Alternatively, default parameters may be used.

Method 400 may proceed to operation 408 during which during which a third stage of sleep may be identified. In various embodiments, the third stage of sleep may be identified based on patterns in measurements indicative of deep sleep. In some embodiments, the third stage may be identified based on electrical measurements, such as those made by an electrode described above. For example, if the electrode detects the presence of slow brain waves such as delta waves interspersed with faster waves, then the third stage may be identified. Accordingly, the identification of electrical oscillatory activity having a designated frequency and designated amplitude may be used to identify the third stage of sleep. In some embodiments, the third stage of sleep is identified based on the identification of the presence of multiple patterns of electrical oscillatory activity. As discussed above, the third stage is identified based on the presence of both slow wave delta waves as well as faster brain waves that have faster frequencies. In various embodiments, the identification of such activity having designated frequencies and designated amplitudes may be used in conjunction with one or more other events, such as previously detected hypnic jerks and slowing of muscular and electrical activity. In this way, a combination of measurements may be utilized to identify the third stage.

In a specific example, identification of the third stage is dependent on the occurrence or detection of the first stage and the second stage, and may occur after and responsive to the identification of the first stage and the second stage. As similarly discussed above, the parameters for detection of the third stage may be configured based on previously generated sleep tracking data generated by the user in previous sleep cycles during a learning phase of use of a tracking device. Alternatively, default parameters may be used.

Method 400 may proceed to operation 410 during which during which fourth stage of sleep may be identified. In various embodiments, the fourth stage of sleep may be identified based on patterns in measurements also indicative of deep sleep. As similarly discussed above, the fourth stage may be identified based on electrical measurements as well as muscular measurements. For example, if the electrode detects the presence of slow brain waves such as delta waves, little to no other brain waves, as well as the absence or minimal presence of muscular activity, then the fourth stage may be identified. As similarly discussed above, identification of the fourth stage may also be dependent on the occurrence or detection of the first stage, the second stage, and the third stage, and may occur after and responsive to the identification of the first stage, the second stage, and the third stage. As similarly discussed above, the parameters for detection of the fourth stage may be configured based on previously generated sleep tracking data generated by the user in previous sleep cycles during a learning phase of use of a tracking device. Alternatively, default parameters may be used.

Method 400 may proceed to operation 412 during which REM sleep may be identified. In various embodiments, REM sleep may be identified based on patterns in electrical and muscular measurements. In some embodiments, REM sleep may be identified based on increases in muscular movements in contraction, increases in blood pressure and heart rate, and low muscle tone. For example, if the accelerometer and sensor detect increases in muscular contraction as well as increases in heart rate following the fourth stage of sleep, then REM sleep may be identified. In a specific example, the onset of such increased in muscular contraction and heart rate may be combined with the detection of a reduction in delta wave activity to identify REM sleep.

As similarly discussed above, identification of REM sleep may also be dependent on the occurrence or detection of the first stage, the second stage, the third stage, and the fourth stage, and may occur after and responsive to the identification of the first stage, the second stage, the third stage, and the fourth stage. As similarly discussed above, the parameters for detection of REM sleep may be configured based on previously generated sleep tracking data generated by the user in previous sleep cycles during a learning phase of use of a tracking device. Alternatively, default parameters may be used.

Method 400 may proceed to operation 414 during which parameters may be updated. Accordingly, measurements made during a sleep cycle may be used to update parameters and thresholds used to identify stages of sleep of a user. For example, measurements, such as peak amplitudes, may be measured with previously generated data and data of other users. Moreover, a report may be generated. Accordingly, a processing device may collect the sleep tracking data during a period of sleep and may generate a report based on the sleep tracking data. The report may identify an amount of time spent in each stage of sleep. Such information may be displayed in a graphical display, such as a time series or chart which illustrates the data and stages of sleep over time.

FIG. 5 illustrates an example of yet another sleep tracking method, configured in accordance with some embodiments. As discussed above, sleep tracking data may be measured and aggregated from various different users to obtain millions of hours of sleep tracking data, and may be utilized to implement one or more operations to provide services to such users. Accordingly, method 500 may commence with operation 502 during which sleep tracking data may be retrieved from a plurality of tracking devices associated with a plurality of users. As discussed above, tracking devices may generate sleep tracking data as a user sleeps, and such data may be aggregated from thousands or millions of users. Moreover, additional data may also be retrieved, such as other user data associated with the users, as will be discussed in greater detail below.

Method 500 may proceed to operation 504 during which a plurality of data objects may be generated based on the retrieved sleep tracking data. In various embodiments, the data objects may be metadata objects. For example, the data objects may represent averaged user data, and averaged numbers of amounts of time spent in particular stages of sleep across a group of users. In some embodiments, the generation of such data object may be configured by an entity, such as an organization or a company.

Method 500 may proceed to operation 506 during which one or more healthcare operations may be identified based on the sleep tracking data. In various embodiments, such healthcare operations may include timing healthcare procedures and treatments, configuration of such treatments, and control of dietary and nutritional needs. For example, if an individual is not getting more than 6-hours of sleep, on average, during the 5 days leading up to a flu shot (immunization for the influenza virus), the individual will produce less than half of the normal antibody response, rendering the vaccine markedly ineffective. Accordingly, sleep tracking data may be used to identify when a flu shot, or other immunizations or vaccines, is appropriate and not appropriate. Furthermore, as will be discussed in greater detail below, messages and/or notifications which may be automated may be generated to notify the user and the user's healthcare professional of such identified timing windows. In another example, an appropriate time for a patient's discharge from a hospital may be identified based on the sleep tracking data. In yet another example, a user's pain sensitivity may be identified based on the sleep tracking data as pain sensitivity may be inversely proportional to amount of sleep. Accordingly, such sleep tracking data may be used to determine a pain management strategy for a user.

Moreover, sleep tracking data may be used to identify and implement a weight management plan as decreased sleep may be correlated with increase weight gain and blood pressure. In some embodiments, when a user's body is sleep deprived, the body may become resistant to letting go of fat, and hormonal imbalances may arise, such as with hormones that tell you to stop eating (leptin) and tells you that you are hungry (ghrelin). Accordingly, sleep tracking data may be integrated with additional data identifying a user's weight management plan, and a system component may generate one or more notifications or messages, or may make one or more modifications to the user's weight management plan. In another example, such sleep tracking data may be used to implement environmental controls, such as the regulation of the light environment for infants. Accordingly, various operations may be identified and implemented based on the sleep tracking data.

Method 500 may proceed to operation 508 during which one or more performance metrics may be identified based on the sleep tracking data. In various embodiments, operational performance, as may be monitored in corporate and military environments, may be proportional to amounts of sleep received by a user. Accordingly, sleep tracking data may be used to predict decreases or increases in performance of a user, as well as implement changes in a schedule or routine associated with the user. For example, if a user's sleep tracking data indicates the user is getting less than a threshold amount of time of a particular stage of sleep, a system component may predict decreased performance, and recommend a change in schedule, routine, or other tasks assigned to the user to enable the user to get more sleep and mitigate the anticipated decrease in performance.

Method 500 may proceed to operation 510 during which one or more additional operations may be identified based on the sleep tracking data. In various embodiments, such additional operations may include identifying and implementing financial constraints and/or operations. In some embodiments, a user may become more impulsive when sleep deprived. In such situations, the user may also be more risk taking, and more sensation seeking. Accordingly, such sleep tracking data may be used to set financial spending limits. In some embodiments, such sleep tracking data may be used to target marketing to a user.

FIG. 6 illustrates an example of a processing device that can be used with various embodiments of the present invention. For instance, the processing device 600 can be used to implement one or more processing and/computing devices according to various embodiments described above, such as processing device 110, mobile device 203, and remote computing device 204. Accordingly, the processing device 600 shown can represent a computing system on a mobile device or on a traditional computer or laptop, etc. According to particular example embodiments, a device 600 suitable for implementing particular embodiments of the present invention includes a processor 601, a memory 603, an interface 611, and a bus 615 (e.g., a PCI bus). The interface 611 may include separate input and output interfaces, or may be a unified interface supporting both operations. When acting under the control of appropriate software or firmware, the processor 601 is responsible for such tasks such as acquisition of measurements and identification of sleep stages. Various specially configured devices can also be used in place of a processor 601 or in addition to processor 601. The complete implementation can also be done in custom hardware. The interface 611 is typically configured to send and receive data packets or data segments over a network. Particular examples of interfaces the device supports include Ethernet interfaces, frame relay interfaces, cable interfaces, DSL interfaces, token ring interfaces, and the like.

In addition, various very high-speed interfaces may be provided such as fast Ethernet interfaces, Gigabit Ethernet interfaces, ATM interfaces, HSSI interfaces, POS interfaces, FDDI interfaces and the like. Generally, these interfaces may include ports appropriate for communication with the appropriate media. In some cases, they may also include an independent processor and, in some instances, volatile RAM. The independent processors may control such communications intensive tasks as packet switching, media control and management.

According to particular example embodiments, the device 600 uses memory 603 to store data and program instructions and maintain a local side cache. The program instructions may control the operation of an operating system and/or one or more applications, for example. The memory or memories may also be configured to store received metadata and batch requested metadata.

Because such information and program instructions may be employed to implement the systems/methods described herein, the present invention relates to tangible, machine readable media that include program instructions, state information, etc. for performing various operations described herein. Examples of machine-readable media include hard disks, floppy disks, magnetic tape, optical media such as CD-ROM disks and DVDs; magneto-optical media such as optical disks, and hardware devices that are specially configured to store and perform program instructions, such as read-only memory devices (ROM) and programmable read-only memory devices (PROMs). Examples of program instructions include both machine code, such as produced by a compiler, and files containing higher level code that may be executed by the computer using an interpreter.

Additional details regarding various embodiments are now discussed. Addressing issues discussed herein may involve two steps. First, we must understand why the problem of sleep deficiency seems to be so resistant to change, and thus persists and grows worse. Second, set against that informative backdrop, we must develop a structured model for affecting change at every possible leverage point we can identify.

First, to an understanding of why the problem persists. Many sleep researchers have likened society's willing acceptance of inadequate sleep to that which we afforded smoking 30 years ago. At the time, researchers held clear evidence linking cigarette smoke to numerous illnesses, cancer chief among them. However, governments failed to implement preventative health policies until it was far too late. Millions of people over many decades suffered mortal consequences; all the while billboard advertisements and TV commercials continued to flaunt the sophisticated and desirable nature of smoking. And so history appears to repeat itself, this time swapping cigarettes for sleep deprivation.

Yet, the analogy is both insufficient, as it vastly undersells the true impact of our lack of sleep, and unhelpful, since it does not explain why we continue to sleep so little. A comparison of the two shows why the former is true. Smoking bears a terrible cost, of course. The chief medical problems are lung disease and cancer, together with increased risk of heart disease, stroke, macular degeneration and asthma. Sleep loss and sleep disorders, on the other hand, are linked to far worse morbidity and mortality, as we have learned. Causal links exist with cancer, heart disease and stroke, but also diabetes, immune deficiency, infection, obesity, death and maiming from vehicle accidents, depression, anxiety, suicide, and Alzheimer's disease. Add to this the unique professional, educational and societal costs that sleep loss imposes, and the gravitas of the problem becomes fully rendered.

Conventional research has been limited in its ability to clearly and comprehensively communicate the hard facts of climate change to the public, and the same is true for sleep. It feels to many that the effects of climate change are far in the future, as is true for the consequences of our own lack of sleep in the now. Related, the consequences of climate change unfold slowly over time, unlike the immediate effects of a natural disaster, such as an earthquake, tsunami or hurricane. As a result, we are far less conscious of climate change, and thus far less willing to accept it, or if accepted, do something about it. Short of a fatal car accident caused by a microsleep, the effects of chronic sleep loss are often similarly slow to unfold, making it difficult for us to recognize the very real contribution of sleep loss to our declining health. Climate change has no consistent or concrete representation. It has no face. In the same way, there is no single image that stands like a visual epitaph for sleep deprivation, unlike a cancerous lung on a packet of cigarettes. Without a poster-child example, the issue of sleep loss, like climate change, feels abstract, evanescent, difficult to connect with, and therefore difficult to rally a cause around.

Many feel that we are more resilient to the effects of sleep loss than those people around us, or at least the consequences will happen to others sooner than they will to ourselves, as is often the case for our belief about being touched by climate change. We find it difficult to accept that the effects of sleep loss—Alzheimer's disease, obesity, diabetes, depression, anxiety, heart attack, etc.—will visit us with the same certainty as others, and even if they do, we do not join the dots and make the connection with our own lifetime lack of sleep.

Regarding Individual Change: Increasing sleep within an individual could be achieved through passive and active methods. Passive solutions are preferable, as they require no effort from the individual. Here are several possibilities, many of which build on proven scientific methods for enhancing sleep quantity and quality.

Many believe the intrusion of technology into our homes and bedrooms is robbing us of precious sleep. Evidence proves this to be true. The future of sleep is about a return to the past in the sense that we must again reunite with a full night of sleep opportunity, every night. But to battle against, rather than unit with, technology is not necessary. We will never put that technological genie back into its bottle, nor do we need to. Instead, technology is part of our current sleep problem, yet disclosed herein is a vision of how it can become part of our sleep savior in the near-term and long-term future.

Within 3-5 years, there will likely be commercially available, affordable devices that accurately track an individual's sleep and circadian rhythm (none currently sold do). When that happens, we can marry these individual sleep trackers with the revolution of in home networked devices, specifically networked home thermostats and network connected home lighting. Two exciting possibilities unfold.

First, such devices could compare the sleep of each individual, in each separate bedroom, night after night, with the temperature sensed in each room by the thermostat. Using common machine-learning algorithms applied over time, we should be able to intelligently teach the home thermostat what the thermal sweet-spot is for each occupant, in each bedroom, based on their measured biophysiology calculated by their individual wearable sleep device, perhaps splitting the difference when there are two or more individuals per room. Granted there are many different factors that make for a good or bad night of sleep, but temperature is very much one of them.

Better still, we could program a natural circadian lull and rise in temperature across the night that is in harmony with each body's expectations. Overtime, we could therefore curate a tailored thermal sleep environment, personalized to the individual occupant(s) of each bedroom, departing from the unhelpful constant thermal environment that plagues the sleep of most people using standard home thermostats. Both these changes require no effort from an individual, and should hasten the speed of sleep onset, increase total sleep time, even deepening NREM sleep quality for all household members, based on the evidence we have previously discussed.

The second passive solution concerns electric light. Many of us suffer from overexposure to nighttime blue-dominant LED light from our digital devices, which suppresses melatonin and delays our sleep timing. Soon, we should be able to engineer LED bulbs with filters that can vary the wavelength of light that they emit, from warm, yellow colors less harmful to melatonin, to strong blue light, that powerfully suppresses it.

Paired with a wearable sleep monitor that can accurately characterize our personal daily rhythms, we can then install these new bulbs throughout a home, with the wearable and the light bulbs in each room being connected to the home network. Communicating together, we can gradually dial-down the harmful blue light as the evening progresses, based on an individual's (or set of individuals) natural sleep-wake pattern defined by the wearable device. We could do this dynamically and seamlessly, as individuals move from one room to the next in real time, again splitting the difference on the fly across individuals based on the mix of their biophysiology. In doing so, the users own brains and bodies, measured and translated through the wearables to the networked home, would synergistically regulate light and thus melatonin release that promotes, rather than impedes, optimal regulation of sleep for one and all.

This is only half of the solution benefit, however. We can saturate our morning indoor environments with powerful blue light that shuts off any lingering melatonin, helping us wake up and feel more alert, with a brighter mood, and do so more quickly.

We could even use this same idea to apply a slight nudge in someone's sleep-wake rhythm within a biologically reasonable range (+/−30-40 minutes), should they desire, gradually moving it earlier or later. This would be equally if not more applicable to helping individuals overcome jetlag using light-emitting personal devices that people often travel with—phones, tablets, laptop computers.

Cars could adopt these same lighting solutions to help manipulate alertness during morning commutes. Mornings, especially early mornings, see some of the highest rates of drowsy-related driving accidents. Car cockpits could be bathed in blue light during morning commutes; especially early in the morning. The levels would have to be tempered so as not to distract the driver, or others on the road. But a sweet spot should not be difficult to find considering that one does not need especially bright lux to have a measurable impact of melatonin suppression and enhanced wakefulness. This idea could be particularly helpful in counties in more northern and southern hemispheres during their respective winter mornings. The workplace offers a similar light regulating opportunity for good, and for those lucky enough to have their own office, that lighting rhythm could be custom-fit to that unique occupant.

Astronauts on the International Space Station travelling through space at 17,500 mph, and complete an orbit of the Earth once every 90-100 minutes. As a result, astronauts experience “daylight” for about 50 minutes, and “night” for about 50 minutes. Although astronauts are therefore treated to the delight of a sunrise and sunset 16 times a day, it wreaks utter havoc on their sleep-wake rhythms, causing terrible issues with insomnia and sleepiness. Make a mistake at your job on planet earth, and your boss may reprimand you. Make a mistake in a long metal tube floating in the vacuum of space and with payloads and mission costs in the hundreds of millions, and the consequences can be much, much worse.

To combat this issue, NASA began collaborating with a large electrical company some years ago to create just these types of special light bulbs. The bulbs were to be installed in the space station so as to bathe the astronauts in a much more earth-like cycle of 24-hour light and dark. With regulated environmental light came a superior regulation of the astronauts' biological melatonin rhythms, including their sleep, thereby reducing operations errors associated with fatigue. Numerous companies are now hard at work constructing similar bulbs for a fraction of the cost that will soon be competitive with standard bulbs. When that happens, these and many other possibilities will become a reality.

Educating people about sleep, through books, engaging lectures or television series, can help combat our sleep deficit. Based on an anonymous sleep survey students complete at the start and the end of the course, the lectures that come in-between increase the amount of sleep they report getting 42 minutes per night, on average. Trivial as that may sound, it does translate to 5-hours of extra sleep each week, or 75 extra hours of sleep each semester.

But this isn't enough. Most likely, a large proportion of my students returned to their shorter, unhealthy sleep habits in the years after. Just as describing the scientific dangers of eating junk food leading to obesity rarely leads to patients choosing an apple over a cookie, abstract knowledge alone is not enough. Additive methods are required.

One bolstering addition known to convert a healthy new habit into a permanent way of life is exposure to your own data. Research in cardiovascular disease is a good example. If patients are given tools that can be used at home to track their improving physiological health in response to an exercise plan, such as blood pressure monitors, weighing scales that log body mass index, or spirometry devices that register respiratory lung capacity, compliance rates with rehabilitation programs increase. Follow up with those same patients after a year or even 5, and more of them have remained active and healthier as a consequence. When it comes to the quantified self, seeing is believing that helps ensure long-term adherence to healthy habits.

With wearables that accurately track our slumber soon emerging, we can convert this same quantified-self approach into a quantified-health model regarding sleep. Using smartphones as a central hub to gather an individual's health data from various sources—physical activity count, such as number of steps or minutes and intensity of exercise, to light exposure, temperature, heart rate, body weight, food intake, work productivity, or mood—we can begin interrelating these factors to an individual, revealing to the individual how their sleep is a direct predictor of their health—mental and physical. It is possible that on the nights you slept more, you eat less food, and healthier food, the next day, you could feel brighter, happier and more positive, had better relationship interactions, and felt as though you had accomplished more in less time at work. Moreover, during months of the year when you were averaging more sleep, you were sick less, your weight, blood pressure, and medication use were all lower, even your relationship or marriage satisfaction, as well as sex life, could be better (all of which have proven links to longer sleep, I should add).

Reinforced day after day, month after month, and ultimately year after year, this nudge could change many people's sleep neglect for the better. If this increased your sleep amount by just 15 or 20 minutes each night, the science indicates that it would make a significant difference across the lifespan, and save trillions of dollars within the global economy at the population level, to name but two benefits. It could be one of the most powerful factors in a future vision that shifts from a model of sickcare, which is what we do now, to healthcare; the latter aiming to stave off a need for the former. That is, preventative health practices, rather than medical treatments, which can be considered inefficient responses to sickness that could have been avoided.

In some embodiments, a stance is switched to analytics i.e., here is your past and/or current sleep and here is your past and/or current body weight, to that of forward-looking, predictolytics. In a related example, there are efforts to create a predictolytics app that starts with you taking a picture of your own face with the camera of your smartphone. Then, the app asks you how many cigarettes you smoke on average a day. Based on scientific data that understands how smoking quantity impacts outward health features such as bags under your eyes, wrinkles, psoriasis, thinning hair and yellowed teeth, the app predictively modifies your face on the assumption of your continued smoking, and does so at different future time points: 1-year, 2-years, 5-years, 10-years, and shows this change relative to the maintenance of your looks if you quit today.

The very same approach could be adopted for sleep, but at many different levels, outward appearance inward brain and body health. For example, we could offer individuals a risk assessment of their Alzheimer's disease trajectory if they continue sleeping too little. Or their cancer risk. Men could explore how much their testicles will shrink, or their testosterone level will drop. We could offer risk predictions for gains in body mass, diabetes risk, or immune resilience. We can then show predictive changes or how improve sleep will reduce these risks and unwanted effects, helping people maintain their goals.

We could also offer people a prediction of when they should or should not get their flu shot based on sleep amount in the week prior. In doing so, it would help maximize their immunity, maximize overall herd immunity strength across a community, and maximize the cost-effectiveness of the treatment offered by healthcare providers, lowering infection rates, hospitalizations and secondary health (and dollar) costs in the later flu season. Considering that the sleep you've had in the week before can alter the success of immunization between 25-60%, depending on the sleep deficit, this simple suggestion could save tens of millions of dollars in direct medical costs. Add to this the price of sleeplessness to the U.S. economy caused by lost productivity and accidents, and current estimates peg the net cost at more than 1 trillion dollars annually. A similar impact on relative gross domestic product caused by this sleep-deprivation tax has been reported in France, Germany, Australia and the United Kingdom.

Educational Change: Over the past 5 weeks, an informal survey was conducted on colleagues, friends and family in this (the United States) country, and in my home country of the United Kingdom. Friends and colleagues were also sampled from Spain, Greece, Australia, Germany, Israel, Japan, South Korea and Canada.

Respondents were asked about the type of health and wellness education they received at school when they were growing up. Did they receive instruction from teachers or specialists on diet? 98% of them did, and many still remembered the details (even if those are changing based on current recommendations). Did they receive tutelage on drugs, alcohol, safe sex and reproductive health? 87% said yes. Was the importance of exercise impressed upon them at some point during their schooling, and/or was the practice of physical education (PE) activities mandatory on a weekly basis? Yes-100% of people confirmed it was. Some form of dietary, exercise and health-related schooling appears to be part of a worldwide educational plan that most children in developed nations receive.

When this set of individuals was asked if they have received any education about sleep, the response was equally universal in the opposite direction: 0% of survey respondents received any educational materials or information about sleep. Even in the health and personal wellness education that some individuals described, there was nothing resembling superficial lip service to sleep's physical or mental health importance. If these individuals are even vaguely representative, it suggests that sleep holds no worldwide place in the education of our children. Generation after generation, our young minds continue to remain unaware of the immediate dangers and protracted health impact of insufficient sleep.

An educational module could be developed that can be implemented in schools around the world. It could take many forms, based on age group—an animated short, accessible online, a board game in physical or digital form, one that could even be played internationally with sleep pen-pals, or a virtual environment that helps you explore the secrets of sleep. There are many options, all of them trans-national.

The goal would be twofold: change the lives of those children, and by way of raised sleep awareness and better sleep practice, have that child pass on their healthy sleep values to their own children. In this way, we would begin a familial transmission of sleep appreciation from one generation to the next, as we do with good manners and morality. Medically, our future generations would not only enjoy a longer life-span, but more importantly, a longer health-span, absolved of the mid- and late-life diseases and disorders that we know are caused by (and not simply associated with) chronic short sleep. The price of delivering such sleep education programs would be a tiny fraction of that we currently pay for our unaddressed global sleep deficit.

Organizational Change: Below are three rather different examples for how we could achieve sleep reform in the workplace and key industries; two short, and one with considerable downstream, knock-on benefits.

First, to employees in the workplace. Jason Fried is a software company co-founder based out of Chicago. He has floated the idea of offer employees bonuses for getting an 8-hour sleep opportunity each night. Granted this requires not only the future development of accurate personal sleep trackers, but employees being motivated to share their data with the company. Nevertheless, when incentivized, and when the reasons for such requests are ethically sound, it seems tenable.

Developing a new business culture that takes care of the entire life cycle of an employee has just as much economic potential as it does compassion. What Fried knows is that when you adopt an employee-centric stance that includes attention to sleep, and scale that up across tens, hundreds or thousands of employees across a company, the net outcome, or dividend return on the sleep investment, for productivity, creativity, work enthusiasm, energy, efficiency, not to mention happiness leading to people wanting to work at your institution, and stay, overrides all misconceptions about grinding down employees with 16-18 hour work days, burning them out in a model of disposability and declining productivity, littered with sick days, all the while triggering low morale and high turnover rates.

Rather than, or as an alternative to, providing financial bonuses, we could offer added vacation time. Individuals who are earning above a basic life-necessity pay grade appear to value time off as much as modest financial perks. A sleep credit system may be implemented, with the sleep time being exchanged for either financial bonuses or, with enough credit, extra vacation days. There would be at least one provisos. The sleep credit system would not simply be calculated on total hours clocked during one week or one month. As we have learned, sleep continuity—consistently getting 7-9-hours of sleep opportunity each night, every night, without running a debt during the week and hoping to pay it off by binge-sleeping at the weekend—is just as important as total sleep time if you are to receive the mental and physical health benefits of sleep. Thus, your “sleep credit score” would be calculated based on a combination of sleep amount and night-to-night sleep continuity.

The second change-idea concerns flexible work shifts. Rather than an off-on-off model of business hours with relatively hard boundaries, business need to adapt a far more tapered vision of hours of operation, one that resembles a squished inverted-U shape. Everyone would be around during a core window for key meetings and coalescence—say 12-3 pm, but with long flexible tail-ends either side to accommodate all chronotypes of circadian rhythms.

Owls could start late (e.g. noon) and work later into the evening, giving their full force of mental capacity and physical energy to their job, and larks likewise with early start and early finish times, preventing them from having to coast the final hours of the “standard” work day with inefficient sleepiness. There are also secondary benefits, not least of which is that it would significantly reduce rush-hour traffic loads morning and evening, the indirect cost-savings of which would, in and of themselves, be non-trivial. Maybe your workplace claims to offer some degree version of this. However, in various consulting experience, that opportunity is suggested, but rarely embraced as acceptable, especially in the eyes of managers and leaders. Dogmas and mindsets appear to be one of the greatest rate-limited factors to building better i.e., sleep-smart, businesses.

The third example of sleep change within industry concerns medicine. Adding to the dire need for injecting more sleep in residents work schedules is a radical rethinking of sleep in patient care. An additional two examples are provided.

### Example 1

Pain: The less sleep an individual has, or the more fragmented their sleep, the more sensitive they become to pain of all kinds. The most common place where people experience significant and sustained pain is often the very last place they can find sound sleep: a hospital. If you have been unfortunate enough to spend even a single night in hospital, you will know this all too well. The problems are especially compounded in the intensive care unit where the most severely sick are cared for i.e., those most in need of sleep's help. Incessant beeping and buzzing from equipment, layered atop of sporadic sound alarms, paired with frequent checks and tests from nurses and doctors, prevents anything like sound sleep for the patient.

Occupational health studies of in-patient rooms and wards report a decibel level of sound pollution that is equivalent to that of a noisy restaurant or bar, around the clock. Indeed, 50-80% of all intensive care alarms are unnecessary or ignorable by staff. Frustratingly, not all tests and patient checkups are time sensitive, yet many are ill-timed with regards to sleep, either during afternoon times when patients would otherwise be enjoying a natural, biphasic-sleep nap, or during early morning hours when patients are only now settling into solid sleep after the challenge of initiating or maintaining it in the noisy, unfamiliar ICU environment.

Little surprise that across cardiac, medical, and surgical intensive care units, systematic studies report uniformly bad sleep in patients. Sleep in the UCI takes longer to initiate, is littered with awakenings and therefore fragmented, more shallow in depth, and contains less REM sleep, and many of these features are significantly triggered by the noise and interrupting environmental factors in the ICU. Worse still, doctors and nurses consistently over-estimate the amount of sleep they think patients obtain in intensive care units, relative to objective assessments of sleep that have been recorded in these individuals. All told, the sleep environment, and thus sleep amount, of a patient in this hospital environment is entirely antithetical to their convalescence.

It should be possible to design a system of medical care that places sleep at the center of patient care, or very close to it. It has been discovered that pain-related centers within the human brain are 62% more sensitive to a painful stimulus when they are sleep deprived. If you give sleep back in full, allowing participants a healthy 8-hour sleep opportunity, their pain tolerance is far higher. It is interesting to note that the pain centers that a full night of sleep was able to palliatively quiet down are the very same brain regions that narcotic pain medications, such as morphine, act upon. Sleep appears to be a natural analgesic, and without it, pain is perceived more acutely by the brain, and, most importantly, felt more powerfully by the individual.

As beneficial are the trickledown effects that would emerge from prioritizing in-patient sleep. Extrapolating from the findings of my own research, we should be able to reduce the dose of narcotic drugs on our hospital wards. This alone would trump any nuisance associated with restructuring medical practice to increase patients' sleep. Morphine is far from a desirable medication. It has safety issues related to the cessation of breathing, dependency and withdrawal, and terribly unpleasant side effects. These include nausea, loss of appetite, cold sweats, itchy skin, urinary and bowel issues, not to mention a form of sedation that prevents natural sleep generation. Morphine further alters the action of other medications, resulting in undesirable interaction effects.

Prescribe better sleep conditions in hospitals; we may find ourselves prescribing significantly lower doses of morphine. In turn, this would lower safety risks, reduce the severity of side effects and decrease the potential for other drug interactions. Reducing the pain medication amounts by increasing sleep may also reduce poly-pharmacy—an non-trivial issue that includes prescribing added medications to offset the side-effects caused by primary drugs, of which morphine is a classic example.

More than just a minimizing of drug doses and number by improving sleep conditions for patients would be a boosting of their immune system so that they could mount a far more potent battle against infection from disease or following surgery, and accelerate postoperative wound healing. The weight of evidence suggests that enhancing sleep quality of patients by even modest amounts should return significant strength to immune response of all kinds, potentially decreasing mortality and morbidity rates. With hastened recovery rates come shorter in-patients stays, reduced healthcare costs and thus reduced insurance rates.

We can start by removing any equipment and alarms that are not necessary for any one patient. Next, we must educate doctors, nurses and hospital administrations on the scientific health benefits of sound sleep, helping them realize the premium we must place on patients slumber. We can also ask patients about their regular sleep schedules on the standard admission form, and then structure assessments and tests around that their habitual sleep-wake rhythms as much as possible.

Being more thoughtful about when we dose patients with certain drugs will also help. When possible, drugs known to disrupt sleep—such as blood-pressure medications or statins—should be given in the morning, rather than the evening. As standard, all patients should be provided earplugs and a facemask when they first come onto a ward, just like the complimentary air travel bag you are given on long-haul flights containing similar equipment. We can also employ dim lighting at night and bright lighting during the day, thereby help to maintain a strong circadian rhythm in patients, and thus a strong wake-sleep pattern.

### Example 2

Neonates: To keep a preterm baby alive and healthy is a perilous challenge. Instability of body temperature, respiratory stress, weight loss and high rates of infection can lead to cardiac instability, neurodevelopment impairments and death. At this premature stage of life, these infants should be sleeping the vast majority of time, both day and night. However, in most neonatal intensive care units, strong lighting will often remain on throughout the night, while a blinding amount of electric overhead light assaults the thin eyelids of these infants during the day. Imagine trying to sleep in constant light for 24-hours a day. Unsurprisingly, infants do not sleep normally under these conditions. It is worth reiterating that which we learned in the chapter on the effects of sleep deprivation in humans and rats: a loss in the ability to maintain core body temperature, cardiovascular stress, respiratory suppression, and a collapse of the immune system.

Sleep stability, time and quality all can be improved. Consequentially, fifty to sixty percent improvements in weight gain and significantly higher oxygen saturation levels in blood were observed, relative to those preterms who did not have their sleep prioritized and thus regularized. The better-sleeping pertem babies were also discharged from the hospital 5 weeks earlier!

We can also implement this strategy in third-world countries without the need for costly lighting changes by simply placing a darkling pieces of plastic—a light-diffusing shroud, if you will—over neonatal cots that costs less than $1, but will have a significant, lux-reducing, benefit to sleep. Even something as simple as bathing a young child at the right time pre-bed (rather than in the middle of the night, as I've seen can occur), would help foster, rather than perturb, good sleep. Both are globally viable methods.

Regarding Societal Change: In the U.S., many healthcare providers offer a financial credit to their members for joining a gym. Insurance agencies could approve valid commercial sleep tracking devices that individuals commonly own. You, the individual, could then upload your sleep credit score to your healthcare provider profile. Based on a tiered, pro-rata system, with reasonable threshold expectations for different age groups, you would be awarded lower insurance cost with increasing sleep credit on a month-to-month basis. Like exercise, this in turn will help improve societal health, en masse, lower the cost of healthcare utilization, allowing people to have longer and healthier lives.

Even with lower insurance paid by the individual, health insurance companies would still gain, as it would lower the cost-burden of their insured individuals, allowing for greater profit margins. Everyone wins. Of course, just like a gym membership, some people will start off adhering to the regime but discontinue, and some may look for ways to bend or play the system regarding accurate sleep assessment of the individual in question. However, even if only 50-60% of individuals truly increase their sleep amount, it could save tens or hundreds of millions of dollars in terms of health costs, not to mention, hundreds of thousands of lives.

While the present disclosure has been particularly shown and described with reference to specific embodiments thereof, it will be understood by those skilled in the art that changes in the form and details of the disclosed embodiments may be made without departing from the spirit or scope of the invention. Specifically, there are many alternative ways of implementing the processes, systems, and apparatuses described. It is therefore intended that the invention be interpreted to include all variations and equivalents that fall within the true spirit and scope of the present invention. Moreover, although particular features have been described as part of each example, any combination of these features or additions of other features are intended to be included within the scope of this disclosure. Accordingly, the embodiments described herein are to be considered as illustrative and not restrictive.

What is claimed is:

1. A system comprising:
a tracking device configured to generate sleep tracking data, the tracking device comprising:
an accelerometer configured to measure macro and micro sensitivity movement of a head, movement and throb of vessels close to an ear, monitor changes in heart rate, pulse rate, and blood pressure of a user;
a sensor configured to measure changes in a middle ear muscle, wherein the sensor is a pressure sensor configured to measure ear drum pressure differentiation as a measure of middle ear muscle activity; and
an electrical sensor configured to measure a voltage of electrical activity from a brain, and identify and measure brain wave patterns;
a processing device configured to identify at least one of a plurality of stages of sleep based on the sleep tracking data generated by the tracking device.

2. The system of claim 1, wherein the accelerometer, the sensor, and the electrical sensor are included in a housing having a shape configured based on an ear of a user.

3. The system of claim 1, wherein the accelerometer is configured to be positioned adjacent to an ear of a user.

4. The system of claim 1, wherein the sensor is a mechanical sensor, and wherein the sensor is configured to be positioned adjacent to an ear of a user.

5. The system of claim 1, wherein the electrical sensor is configured identify the brain wave patterns based on electrophysiological measurements.

6. The system of claim 1, wherein the plurality of stages of sleep comprises a first stage of sleep, a second stage of sleep, a third stage of sleep, a fourth stage of sleep, and rapid eye movement (REM) sleep.

7. The system of claim 6, wherein the at least one of the plurality of stages of sleep is identified based, at least in part, on a plurality of measurements made by the accelerometer, the sensor, and the electrical sensor.

8. The system of claim 6, wherein the at least one of the plurality of stages of sleep is identified based, at least in part, on a plurality of measurements made by the accelerometer, the sensor, and the electrical sensor, and a transition associated with another of the plurality of stages of sleep.

9. The system of claim 1 further comprising:
a communications interface configured to transmit measurements made by the accelerometer, the sensor, and the electrical sensor to the processing device.

10. A device comprising:
an accelerometer configured to measure macro and micro sensitivity movement of a head, movement and throb of vessels close to an ear, monitor changes in heart rate, pulse rate, and blood pressure of a user;
a sensor configured to measure changes in a middle ear muscle, wherein the sensor is a pressure sensor configured to measure ear drum pressure differentiation as a measure of middle ear muscle activity; and
an electrical sensor configured to measure a voltage of electrical activity from a brain, and identify and measure brain wave patterns.

11. The device of claim 10, wherein the accelerometer, the sensor, and the electrical sensor are included in a housing having a shape configured based on an ear of a user.

12. The device of claim 10 wherein the accelerometer is configured to be positioned adjacent to an ear of a user.

13. The system of claim 10, wherein the sensor is a mechanical sensor, and wherein the sensor is configured to be positioned adjacent to an ear of a user.

14. The system of claim 10, wherein the electrical sensor is configured identify the brain wave patterns based on electrophysiological measurements.

15. The device of claim 14, wherein the electrical sensor comprises an array of sensing elements.

16. The device of claim 10 further comprising:
a communications interface configured to transmit measurements made by the accelerometer, the sensor, and the electrical sensor.

17. A method comprising:
measuring, using an accelerometer, macro and micro sensitivity movement of a head, movement and throb of vessels close to an ear, changes in heart rate, pulse rate, and blood pressure of a user;
measuring, using a sensor, changes in a middle ear muscle, wherein the sensor is a pressure sensor that measures ear drum pressure differentiation as a measure of middle ear muscle activity;
measuring, using an electrical sensor, a voltage of electrical activity from a brain;
identifying, using the electrical sensor, brain wave patterns; and
identifying at least one stage of sleep from a plurality of stages of sleep based on the measurements.

18. The method of claim 17, wherein the plurality of stages of sleep comprises a first stage of sleep, a second stage of sleep, a third stage of sleep, a fourth stage of sleep, and rapid eye movement (REM) sleep.

19. The method of claim 17, wherein the at least one of the plurality of stages of sleep is identified based, at least in part, on a plurality of measurements made by the accelerometer, the sensor, and the electrical sensor.

20. The method of claim 17, wherein the at least one of the plurality of stages of sleep is identified based, at least in part, on a plurality of measurements made by the accelerometer, the sensor, and the electrical sensor, and a transition associated with another of the plurality of stages of sleep.
